SEQUENCE LISTING (19 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 8 aa, Artificial Sequence
  Chemically synthesized peptide fragment
YTSLIHSL

SEQ ID NO: 3 - protein, 15 aa, Artificial Sequence
  Chemically synthesized peptide fragment
YTSLIHSLIEESQNQ

SEQ ID NO: 4 - protein, 16 aa, Artificial Sequence
  Chemically synthesized peptide fragment
YTSLIHSLIEESQNQQ

SEQ ID NO: 5 - protein, 18 aa, Artificial Sequence
  Chemically synthesized peptide fragment
YTSLIHSLIEESQNQQEK

SEQ ID NO: 6 - protein, 7 aa, Artificial Sequence
  Chemically synthesized peptide fragment
IEESQNQ

SEQ ID NO: 7 - protein, 8 aa, Artificial Sequence
  Chemically synthesized peptide fragment
IEESQNQQ

SEQ ID NO: 8 - protein, 20 aa, Artificial Sequence
  Chemically synthesized peptide fragment
QEKNEQELLELDKWASLWNW

SEQ ID NO: 9 - protein, 21 aa, Artificial Sequence
  Chemically synthesized peptide fragment
QEKNEQELLELDKWASLWNWF

SEQ ID NO: 10 - protein, 7 aa, Artificial Sequence
  Chemically synthesized peptide fragment
EKNEQEL

SEQ ID NO: 11 - protein, 10 aa, Artificial Sequence
  Chemically synthesized peptide fragment
EKNEQELLEL

SEQ ID NO: 12 - protein, 20 aa, Artificial Sequence
  Chemically synthesized peptide fragment
EKNEQELLELDKWASLWNWF

SEQ ID NO: 13 - protein, 17 aa, Artificial Sequence
  Chemically synthesized peptide fragment
NEQELLELDKWASLWNW

SEQ ID NO: 14 - protein, 18 aa, Artificial Sequence
  Chemically synthesized peptide fragment
NEQELLELDKWASLWNWF

SEQ ID NO: 15 - protein, 12 aa, Artificial Sequence
  Chemically synthesized peptide fragment
LELDKWASLWNW

SEQ ID NO: 16 - protein, 13 aa, Artificial Sequence
  Chemically synthesized peptide fragment
LELDKWASLWNWF

SEQ ID NO: 17 - protein, 9 aa, Artificial Sequence
  Chemically synthesized peptide fragment
DKWASLWNW

SEQ ID NO: 18 - protein, 10 aa, Artificial Sequence
  Chemically synthesized peptide fragment
DKWASLWNWF

SEQ ID NO: 19 - protein, 19 aa, Artificial Sequence
  Chemically synthesized peptide fragment
EKNEQELLELDKWASLWNW

(19) United States
(12) Patent Application Publication
(10) Pub. No.: US 2005/0165215 A1
(43) Pub. Date: Jul. 28, 2005

(54) Peptide synthesis and deprotection using a cosolvent

(76) Inventors: Roger D. Bigelow, Boulder, CO (US); Mark A. Schwindt, Boulder, CO (US); Gregory P. Withers, Boulder, CO (US)

Correspondence Address: Roche Colorado Corporation, 2075 North 55th Street, Boulder, CO, 80301, US

(21) Appl. No.: 11/021,647

(22) Filed: Dec. 23, 2004

Related U.S. Application Data
(60) Provisional application No. 60/533,710, filed on Dec. 31, 2003.

(51) Int. Cl. C07K 1/02

(52) U.S. Cl. 530/333

(57) ABSTRACT

The invention provides methods for synthesizing peptides, which include taking an organic solvent having non-resin bound protected peptide and performing a deprotection reaction on the non-resin bound protected peptide. In these methods it is not required that the peptide is dried immediately before providing to the deprotection reaction. Also provided are methods of synthesizing peptides, wherein a protected peptide is formed in a solution phase reaction, dissolved into an organic solvent, and then introduced into a deprotection reaction. Also provided are methods of synthesizing peptides, wherein a non-resin bound protected peptide is concentrated in an organic solvent prior to being subject to a deprotection reaction.

### PRIORITY CLAIM

The present non-provisional patent Application claims priority under 35 USC §119(e) from United States Provisional Patent Application having Ser. No. 60/533,710, filed on Dec. 31, 2003, and titled PEPTIDE SYNTHESIS AND DEPROTECTION USING A COSOLVENT, wherein said provisional patent application is commonly owned by the owner of the present patent application and wherein the entire contents of said provisional patent application is incorporated herein by reference.

### FIELD OF THE INVENTION

The present invention relates to the synthesis of peptides and methods for the isolation of peptides during the synthetic process. The invention also relates to improvements for the large-scale synthesis of peptides.

### BACKGROUND

Many methods for peptide synthesis are described in the literature (for examples, see U.S. Pat. No. 6,015,881; Mergler et al. (1988) Tetrahedron Letters 29: 4005-4008; Mergler et al. (1988) Tetrahedron Letters 29: 4009-4012; Kamber et al. (eds), Peptides, Chemistry and Biology, ESCOM, Leiden (1992) 525-526; Riniker et al. (1993) Tetrahedron Letters 49: 9307-9320; Lloyd-Williams et al. (1993) Tetrahedron Letters 49: 11065-11133; and Andersson et al. (2000) Biopolymers 55: 227-250. The various methods of synthesis are distinguished by the physical state of the phase in which the synthesis takes place, namely liquid phase or solid phase.

In some cases, liquid and solid phase peptide synthesis procedures have been scaled up in order to produce peptides on a pilot plant scale or on an industrial scale. Scaled-up, or “large-scale” peptide synthesis procedures are typically performed to produce peptides that have a pharmaceutical utility (Bray, B. L., Nature Rev., 2: 587-593 (2003)). In order to meet the needs of global health care, pharmaceutically useful peptides may require yearly production levels in over kilogram amounts. Due to the technical difficulties and costs associated with the production of peptides, in general, there is a great need to optimize processes associated with production of pharmaceutically useful peptides on a large-scale basis in order to make their widespread use in global health care economically feasible.

The synthesis of a peptide typically requires a multitude of steps, many of which may be performed in one or more pieces of equipment and might be repeated either in succession, in parallel, or in an overlapping manner, during the entire synthetic process. When performing peptide synthesis, one or more small improvements in any particular step can lead to significant gains in the overall cost reduction, time reduction, and quality of the procedure.

Some steps in peptide synthesis have traditionally required treating the peptide, or an intermediate product, with harsh conditions, such as high temperatures or drying. If the peptide, or an intermediate product is sensitive to these conditions, significant degradation can occur during these steps, leading to loss of yield.

For example, prior art teaches the drying of a peptide after it has been formed in a solution phase reaction and in order to prepare it for a deprotection reaction. A deprotection reaction involves the removal of protecting groups from a peptide and can require the removal of impurities from the peptide product, to a certain extent, prior to deprotection. For example, drying steps have been performed before global deprotection, a process where all of the protecting groups, including terminal protecting groups and side chain protecting groups, are removed from the peptide. Steps such as drying that precede a deprotection reaction can affect the quality of the peptide and can also affect aspects of the deprotection reaction.

Therefore, there is a need for improved procedures in peptide synthesis that allow intermediate products to be carried through the stages of the synthetic procedure with minimal degradation and ease. At the same time, it is desirable that these improvements do not significantly compromise the efficacy of steps in the synthetic process.

In particular there is a need for improvements for the synthesis of therapeutically useful peptides, or peptide intermediates thereof, that are relatively long, such as peptides that are longer than 15, 20, or 25 amino acid residues. It is understood that the amount of time and materials involved in synthesizing these long peptides is very considerable. In many cases, due to technical issues, synthesis of these peptides occurs using a combination of solid phase and solution phase steps, typically known as a hybrid approach. Therefore, there is a need for improvements in the synthesis of relatively long peptides, such as peptides made by a hybrid approach in large-scale, pilot plant scale, and small-scale procedures.

### SUMMARY OF THE INVENTION

The present invention relates to methods for synthesizing peptides, in particular methods for the improved processing of a non-resin bound peptide having at least one protecting group. Generally, the methods of the invention teach the use of an organic solvent as an agent that allows the protected peptide to be introduced into a deprotection reaction. At least a portion of the organic solvent is necessarily used as a cosolvent in the deprotection reaction. More specifically, the organic solvent can be used to take the peptide from a solution phase reaction, such as a solution phase coupling reaction, into a deprotection reaction; the process of which provides a number of important advantages in comparison to previously used methods.

One important improvement according to the invention is that there is no need to dry the peptide immediately prior to deprotection. Certain steps that may take place prior to the deprotection can be carried out with the peptide in the organic solvent. Traditional methods often include steps of precipitating and then drying the peptide prior to deprotection, which can expose the peptide to factors such as heat and air that can advance the degradation of the peptide, especially sensitive peptides. In the current methods, organic solvent can be kept at temperatures that greatly minimize these detrimental effects.

In addition, traditional methods that often accompany drying, for example trituration (the crushing of precipitated and dried peptide into small particles in an organic solvent(s)) can be physically harsh on the peptide and difficult to accomplish in a scaled-up processes (multi kg). In the present invention, the peptide is dissolved in the organic solvent and abrasive practices such as trituration can be avoided.

Furthermore, in order to prepare a peptide for a deprotection reaction it is often desirable to separate as many impurities from the peptide as possible. These impurities may originate from, for example, a solution phase coupling reaction that can precede the deprotection reaction. In traditional methods, steps of drying and trituration have been incorporated into a purification process to remove these impurities. This is generally undesirable for a large-scale process. By contrast, methods of the present invention, in some aspects, allow the organic solvent containing the peptide to be subject to one or more wash steps to remove impurities. Washing the peptide in organic solvent with one or more aqueous solution(s), for example, basic and/or acidic solutions, is an effective method for reducing impurities and allows the peptide dissolved in an organic solvent to be used directly in a deprotection reaction.

At least a portion of the original volume of organic solvent having the peptide is taken into the deprotection reaction where it is necessarily present as a cosolvent. This, in fact, provides many advantages in the step of the deprotection reaction and also steps that may follow. For example, this approach allows a deprotection reaction to be carried out in a relatively broad time frame without having the peptide be subject to undue degradation. Furthermore, the cosolvent also permits greater variability in the conditions under which the deprotection reaction is carried out. For example, the cosolvent can lend to the use of lower or higher concentrations of the deprotection reagent, and colder or warmer reaction conditions; these variations can enhance the rate at which the peptide is deprotected. Also, since most or all of the peptide is dissolved in the cosolvent prior to deprotection, the deprotection reaction can be initiated and carried out on the bulk of the peptide in a more uniform manner. This is particularly advantageous in scaled-up processes. This reduces the occurrence of degradation reactions such as delamination and peptide cleavage.

The cosolvent also improves aspects of steps that can be performed after the deprotection reaction. For example, presence of the cosolvent allows quenching of the deprotection reaction to be controlled to a greater extent. This, in turn, improves peptide quality. Also, presence of the cosolvent allows for better filtering and washing of the peptide after the deprotection reaction has been quenched. This, in turn, improves peptide quality and reduces processing time.

In another aspect, the invention provides a method for preparing and deprotecting a peptide that includes steps of forming a peptide having a protecting group in a solution phase reaction; dissolving the protected peptide in an organic solvent to form a peptide composition; and then contacting the peptide composition with a deprotection agent.

In another aspect, the invention provides a method for deprotecting a peptide that includes the steps of concentrating a non-resin bound peptide having a protecting group in an organic solvent to provide a peptide composition; and contacting the peptide composition with a deprotection agent.

### BRIEF DESCRIPTION OF THE DRAWINGS

FIG. 1 illustrates a reaction vessel having a filter decanting system that can be used in accordance with the methods of the present invention.

### DETAILED DESCRIPTION

All publications and patents mentioned herein are hereby incorporated by reference. The publications and patents disclosed herein are provided solely for their disclosure.

The terminology used herein is not intended to limit the scope of the invention. Throughout the text, including the appended claims, the singular forms “a,” “an,” and “the” include plural reference unless the context clearly dictates otherwise. Thus, for example, a reference to “a protecting group” is a reference to one or more protecting groups and includes equivalents thereof known to those skilled in the art. In this invention, certain terms are used frequently, the meanings of which are provided herein. Unless defined otherwise, terms used herein have the same meaning as commonly understood to one of ordinary skill in the art in this field of technology. Some terms may also be explained in greater detail later in the specification.

The present invention provides methods for deprotecting a peptide having a protecting group. These methods can be performed during or after the synthesis of peptides and peptide intermediates. Preferably, the deprotection reaction is able to at least remove side-chain protecting groups from a peptide. The methods include a step of providing a non-resin bound peptide material having a protecting group in an organic solvent and a step of contacting the non-resin bound peptide in the organic solvent with a deprotection agent. “Non-resin bound” refers to the peptide not being coupled to an insoluble support material (for example, resins for solid-phase synthesis). “Peptide material” refers to any peptide or peptide intermediate fragment and is also referred to as “peptide” herein. The methods as described herein can be performed at times during or after solution-phase, solid-phase, or hybrid synthesis procedures.

In some embodiments, the methods described herein are performed in a scaled-up peptide synthesis procedure. Scaled-up procedures are typically performed to provide an amount of peptide useful for distribution. For example the amount of peptide in a scaled-up procedure can be 500 g, or 1 kg per batch or more, and more typically tens of kg to hundreds of kg per batch or more. In scaled-up synthetic procedures such as large-scale synthesis one or more large reaction vessels can be used. These can accommodate quantities of reagents such as resins, solvents, amino acids, and chemicals for various steps in the synthesis process, in a size that allows for production of peptides in amounts, for example, in the range of 100-500 kilograms or more.

Accordingly, the methods described herein are particularly suitable for improving aspects of the peptide synthesis, particularly for scaled-up procedures. Improvements include a reduction in overall processing (synthesis) time and improvements in the yield and purity of intermediates and final peptide products.

The methods described herein can be applied in the synthesis of any peptide, for example, any peptide chain of amino acid residues that are chemically bound together. The amino acid residues of the peptide synthesized can be naturally occurring amino acid residues, non-natural amino acid residues, or combinations thereof. Suitable peptides also include peptide intermediates which include compounds having an amino acid backbone and that are typically subject to one or more subsequent steps in a peptide synthesis scheme.

Peptides used in the methods of the invention can include common and rare naturally occurring L-amino acids, non-natural amino acids, and similar residues that can be incorporated into a peptide.

The twenty common naturally-occurring amino acids residues which are represented by the one-letter symbols as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine). Naturally-occurring rare amino acids include, for example, selenocysteine, and pyrrolysine.

Non-natural amino acids can be organic compounds having a similar structure and reactivity to that of a naturally-occurring amino acid can include, for example, D-amino acids, beta amino acids, gamma amino acids; cyclic amino acid analogs, propargylglycine derivatives, 2-amino-4-cyanobutyric acid derivatives, Weinreb amides of α-amino acids, and amino alcohols.

The methods of the invention can be used for the synthesis of peptides, for example, pharmaceutically useful peptides, that can be made using a solid-phase approach, a solution phase approach, or a combination of approaches. Exemplary pharmaceutically useful peptides include, but are not limited to oxytocin; vasopressin analogues such as felypressin, pitressin, lypressin, desmopressin, terlipressin; atosiban; adrenocorticotropic hormone (ACTH); insulin, glucagon; secretin; calcitonins such as human calcitonin, salmon calcitonin, eel calcitonin, dicarba-eel calcitonin (elcatonin); luteinizing hormone-releasing hormone (LH-RH) and analogues such as leuprolide, deslorelin, triptorelin, goserelin, buserelin; nafarelin, cetrorelix, ganirelix, parathyroid hormone (PTH); human coriticotropin-releasing factor, ovine coriticotropin-releasing factor; growth hormone releasing factor; somatostatin; lanreotide, octreotide; thyrotropin releasing hormone (TRH); thymosin α-1; thymopentin (TP-5); cyclosporin; integrilin; and angiotensin-converting enzyme inhibitors such as enalapril and lisinopril.

In some embodiments, the invention relates to methods for the synthesis of the enfuvirtide peptide (also known as T-20) and peptides having enfuvirtide activity. Enfuvirtide is a peptide, which corresponds to amino acid residues 638 to 673 of the transmembrane protein gp41 from the HIV-1_{L}AI isolate and has the 36 amino acid sequence:
YTSLIHSLIEESQNQQEKNEQELLELDKWASLWNWF (SEQ ID NO 1)

Enfuvirtide is an anti-retroviral drug used for the treatment of HIV-1 infection. Enfuvirtide functions to block fusion of the HIV-1 viral particle with host cells by blocking the conformational changes required for membrane fusion. Peptides having this type of activity are herein referred to as having enfuvirtide activity.

Enfuvirtide synthesis typically utilizes both solid and liquid phase procedures to synthesize and combine groups of specific peptide fragments to yield the enfuvirtide product (Bray, B. L., Nature Rev., 2: 587-593 (2003)). The present invention provides methods for the improved synthesis of both enfuvirtide peptide intermediates and full-length enfuvirtide products. The methods of the invention also include the synthesis of peptides having enfuvirtide activity and peptide intermediates used to prepare peptides having enfuvirtide activity. Peptides having enfuvirtide activity are described in U.S. Pat. Nos. 5,464,933 and 5,656,480, and PCT Publication No. WO 96/19495.

Peptide intermediate fragments that can be used to prepare enfuvirtide include, but are not limited to, those having amino acid sequences as depicted in Table 1 below:

TABLE 1

| | | | |
|---|---|---|---|
| | | | CORRESPONDING NUMBERED |
| PEPTIDE | | | AMINO ACID SEQUENCE |
| NO. | AMINO ACID SEQUENCE | SEQ ID NO | OF T-20 |

| | | | |
|---|---|---|---|
| 1 | YTSLIHSL | (SEQ ID NO:2) | 1-8 |
| 2 | YTSLIHSLIEESQNQ | (SEQ ID NO:3) | 1-15 |
| 3 | YTSLIHSLIEESQNQQ | (SEQ ID NO:4) | 1-16 |
| 4 | YTSLIHSLIEESQNQQEK | (SEQ ID NO:5) | 1-18 |
| 5 | IEESQNQ | (SEQ ID NO:6) | 9-15 |
| 6 | IEESQNQQ | (SEQ ID NO:7) | 9-16 |
| 7 | QEKNEQELLELDKWASLWNW | (SEQ ID NO:8) | 16-35 |
| 8 | QEKNEQELLELDKWASLWNWF | (SEQ ID NO:9) | 16-36 |
| 9 | EKNEQEL | (SEQ ID NO:1O) | 17-23 |
| 10 | EKNEQELLEL | (SEQ ID NO:11) | 17-26 |
| 11 | EKNEQELLELDKWASLWNWF | (SEQ ID NO:12) | 17-36 |
| 12 | NEQELLELDKWASLWNW | (SEQ ID NO:13) | 19-35 |
| 13 | NEQELLELDKWASLWNWF | (SEQ ID NO:14) | 19-36 |
| 14 | LELDKWASLWNW | (SEQ ID NO:15) | 24-35 |
| 15 | LELDKWASLWNWF | (SEQ ID NO:16) | 24-36 |
| 16 | DKWASLWNW | (SEQ ID NO:17) | 27-35 |
| 17 | DKWASLWNWF | (SEQ ID NO:18) | 27-36 |
| 18 | EKNEQELLELDKWASLWNW | (SEQ ID NO:19) | 17-35 |

As indicated, the methods described herein can be used during or after solid phase synthesis, solution phase synthesis, or a hybrid synthesis. In some embodiments, solid phase synthesis can be used to prepare a peptide, or a peptide intermediate, which can be utilized in the methods described herein.

The solid phase synthesis step typically involves a peptide or an amino acid coupled to an insoluble support resin. In solid phase, an amino acid residue is coupled to the resin and additional residues are subsequently coupled to build the peptide chain. For example, solid phase synthesis using Fmoc chemistry is performed to prepare a peptide coupled to a resin. For example, any peptide intermediate fragment of enfuvirtide, such as those listed in Table 1 can be prepared using this method. At some point during solid phase synthesis, the peptide that has been formed is cleaved from the resin. In accordance with the methods of the invention, the step of cleaving provides a peptide having at least side chain protecting groups. After cleaving, the peptide is separated from the resin. After this, the peptide can be subject to a solution phase reaction, such as a coupling reaction to couple the protected peptide to another amino acid or peptide. The peptide can then be dissolved in an organic solvent and subjected to a deprotection reaction.

Methods for the synthesis of peptides using a solid-phase approach are well known in the art. Accordingly, the invention contemplates using any solid phase synthetic approach for preparing a peptide, which can then be used in the methods described herein. See, for example, Carpin et al. (1970), J. Am. Chem. Soc. 92(19): 5748-5749; Carpin et al. (1972), J. Org. Chem. 37(22): 3404-3409, “Fmoc Solid Phase Peptide Synthesis,” Weng C. Chan and Peter D. White Eds; (2000) Oxford University Press Oxford Eng. The solid phase syntheses of the peptide fragment intermediates of the invention can be carried out on an acid sensitive solid support, such as a “Wang” resin, which comprises a copolymer of styrene and divinylbenzene with 4-hydroxymethylphenyloxy-methyl anchoring groups (Wang, S. S. 1973, J. Am. Chem. Soc.). Other suitable resins include 2-chlorotrityl chloride resin (Barlos et al. (1989) Tetrahedron Letters 30(30): 3943-3946), and 4-hydroxymethyl-3-methoxyphenoxybutyric acid resin (Richter et al. (1994), Tetrahedron Letters 35(27): 4705-4706). The Wang, 2-chlorotrityl chloride, and 4-hydroxymethyl-3-methoxyphenoxy butyric acid resins can be purchased from Calbiochem-Novabiochem Corp., San Diego, Calif.

As an alternative to solid-phase synthesis of peptides or peptide fragments, solution-phase synthesis can be used to build a peptide chain. The choice of whether to use solution-phase synthesis or solid-phase synthesis to build a peptide chain can depend on any one or a combination of a number of factors, including, for example, the length of the peptide, the sequence of the peptide, and the amount of peptide desired to be produced. Solution phase synthesis generally involves the coupling, in solution, of an amino acid to another amino acid, the coupling of peptide to amino acid, or the coupling of two peptides together. Typically, in solution-phase synthesis, the amino acids or peptides are not coupled to a resin, as they would be in a solid phase synthesis procedure. For example, a solution phase step can be performed to couple an amino acid to a growing peptide chain and then the formed peptide can be dissolved in a organic solvent and then provided to a deprotection reaction.

The synthesis of peptides and peptide fragments as described above typically involves preparing peptides having one or more side-chain protecting groups. In the later stages of the overall peptide synthesis process these side-chain protecting groups can be removed in a deprotection reaction. According to the invention, a peptide having one or more side-chain protecting groups can be provided in an organic solvent and then contacted with a deprotection agent to remove these side-chain protecting groups.

The side-chain protecting groups are typically chemical moieties coupled to the side chain (R group) of an amino acid and which predominantly prevent a portion of the side chain from reacting with chemicals used in the various steps of peptide synthesis procedures. Examples of side chain protecting groups include acetyl (Ac); benzoyl (Bz); tert-butyl; triphenylmethyl (trityl); tetrahydropyranyl; benzyl ether (Bzl); 2,6-dichlorobenzyl (DCB); t-butoxycarbonyl (BOC); nitro; p-toluenesulfonyl (Tos); adamantyloxycarbonyl; xanthyl (Xan); benzyl; methyl; ethyl; t-butyl ester; benzyloxycarbonyl (Z); 2-chlorobenzyloxycarbonyl (2-CI-Z); Tos; t-amyloxycarbonyl (Aoc); aromatic or aliphatic urethan-type protecting groups; photolabile groups such as nitro veritryl oxycarbonyl (NVOC); and fluoride labile groups such as trimethylsilylethyl oxycarbonyl (TEOC).

Commonly used side chain protecting groups include t-Bu group for Tyr(Y), Thr(T), Ser(S), and Asp(D) amino acid residues; the trt group for His(H), Gln(O), and Asn(N) amino acid residues; and the Boc group for Lys(K) and Trp(W) amino acid residues.

The methods described herein can be integrated into the synthesis of peptides having, for example, different amino acid sequences, different lengths, and different chemical modifications. That is, it is believed that any non-resin bound peptide that includes a protecting group can be provided in an organic solvent and then introduced into a deprotection reaction as described herein. In particular, the methods are useful when for supplying a full-length peptide in an organic solvent to a deprotection reaction. A “full-length peptide” refers to a peptide that is complete in regards to its amino acid content, that is, a peptide product that contains a desired number of amino acids. Full-length peptides include mature peptide products, for example, peptides wherein no additional chemical modifications are desired, or peptide products wherein certain chemical (non-amino) acid modifications can be made. For example, a full-length peptide can have protecting groups, such as side chain protecting groups as described. A full-length peptide can be obtained, for example, after the solid-phase synthesis and/or solution-phase synthesis to form a peptide that has a desired number of amino acid residues.

After the steps of preparing a full-length peptide are completed, the full-length peptide can still have protecting groups. These protected full-length peptides can then be provided in an organic solvent and subject to a deprotection reaction to remove the protecting groups. Following peptide deprotection, the peptide can be isolated or purified. If necessary, the peptide can be subject to one or more processing steps to remove unwanted chemical groups or to add desired chemical groups.

By way of example, the following text illustrates how the methods of providing a peptide material having a protecting group in an organic solvent to a deprotection reaction can fit into an overall peptide synthesis process. A hybrid approach for the synthesis of a peptide is set forth to represent a suitable overall synthetic process. A more detailed description of steps involved in this synthetic process is set forth herein.

(1) Peptide intermediate fragments are coupled together in solution to form a full-length peptide (the peptide intermediate fragments can be prepared according to the solid phase or solution-phase procedures as described above). (2) The full-length peptide is then precipitated from the coupling reaction with a suitable precipitating agent, such as water or a non-solvent. (3) Next, an organic solvent, such as DCM, is added to the precipitated peptide wherein the precipitated peptide is extracted into the organic solvent, where it is dissolved or suspended. The precipitating agent separates from the organic solvent and is removed, leaving the peptide in the organic solvent. (4) The organic solvent containing the peptide can then be washed one or more times with the precipitating agent to remove, for example, solvents or reagents from the coupling reaction. Washing can be performed as necessary to provide a peptide/solvent mixture having a desired degree of purity. (5) Optionally, the organic solvent can be concentrated by distillation, or any suitable technique, to provide the peptide at a higher concentration in the organic solvent. (6) The resulting concentrated peptide composition is then subject to a deprotection reaction to remove protecting groups on the peptide.

Additional steps can be carried out, for example, precipitation and isolation of the peptide, additional chemical modification, such as decarboxylation (if needed), and final purification. These aspects of the invention are also discussed in greater detail herein.

It is understood that one, some, or all of the following steps can be performed in a particular reaction vessel. In other cases the following steps can be performed in separate reaction vessels if desired.

The following text describes the preparation of a peptide using a hybrid synthesis approach and includes description of the steps of providing a peptide having protecting groups in an organic solvent and then providing this to a deprotection reaction.

Peptide intermediate fragments are coupled together in solution to form a full-length peptide. As starting materials for a coupling reaction, two peptide intermediate fragments, or more, are provided. These peptide intermediate fragments can be prepared according to the solid phase or solution-phase procedures as described herein. The peptide intermediate fragments include side chain protecting groups, if needed, and are chemically arranged wherein the N-terminus of one fragment can be coupled to the C-terminus of the other fragment, or vice-versa. Preferably, the peptides are supplied to the coupling reaction at a purity level of 80% or greater, or more preferably 85% or greater based on a HPLC profile. Peptide coupling reactions are reviewed in, for example, New Trends in Peptide Coupling Reagents; Albericio, Fernando; Chinchilla, Rafeal; Dodsworth, David J.; and Najera, Armen; Organic Preparations and Procedures International (2003), 33(3), 203-303.

Coupling can be carried out using in situ coupling reagents, for example, BOP, o-(benzotriazol-1-yl)-N,N,N′,N′-tetramethyluronium hexafluorophosphate (HBTU), HATU, dicyclohexylcarbodiimide (DCC), water-soluble carbodiimide (WSCDI), or o-(benzotriazol-1-yl)-N,N,N′,N′-tetramethyluronium tetrafluoroborate (TBTU). Other coupling techniques use preformed active esters such as hydroxysuccinimide (HOSu) and p-nitrophenol (HONp) esters; preformed symmetrical anhydrides; N-carboxyanhydrides (NCAs); or acid halides such as acyl fluoride as well as acyl chloride.

A suitable coupling solvent can be used in the coupling reaction. It is understood that the coupling solvent(s) used can affect the degree of racemization of the peptide bond formed; the solubility of the peptide and/or peptide fragments; and the coupling reaction rate.

In some embodiments, the coupling reaction includes a water-miscible solvent(s). Examples of a water-miscible solvent include, for example, DMSO, pyridine, chloroform, dioxane, tetrahydrofuran, ethyl acetate, N-methylpyrrolidone, dimethylformamide, dioxane, or mixtures thereof.

In other embodiments, the coupling reaction includes a non water-miscible solvent. An exemplary non water-miscible solvent is chloroform. In these embodiments, the non water-miscible solvent is preferably compatible with the deprotection reaction; for example, if a non water-miscible solvent is used preferably it does not adversely affect the deprotection reaction.

The reagents can be combined in a suitable order. For example, the peptide intermediate fragments can be dissolved in the coupling solvent along with the coupling reagent. During or after the process of dissolving the peptide intermediates in the coupling solvent, the mixture can be chilled, for example, to a temperature of below 20° C., or below 0° C. Additional reagents such as a tertiary base and a racemization suppressant can be added. The reaction can then be warmed, if desired. For example, the temperature can be warmed up to a temperature between 0° C. to 30° C. over the period of the reaction. The reaction can be performed at one or over a range of temperatures until a desired amount of product is formed. Typical reaction times are 2 hours or greater. The reaction can be monitored to determine the extent of coupling, for example, by BPLC or by Kaiser test.

After a desired about of peptide product has formed from the coupling of the peptide intermediate fragments, the coupling reaction can be quenched by addition of a precipitating reagent. One cost-effective precipitating agent is water. In other cases a non-polar solvent, for example, hexaneisooctane, cyclohexane, methylcyclohexane, can be used to precipitate the peptide; however, generally, a precipitating solvent is used that is miscible with the coupling solvent.

The precipitating agent can be added in an amount sufficient to effectively remove the coupling solvent. In one embodiment, the precipitating agent is preferably added in an amount of at least 1:1 relative to the coupling reaction solvent.

After the peptide has precipitated, an organic solvent can be added to the mixture having the precipitated peptide. During this step the peptide is extracted into and dissolved or suspended in the organic solvent. The mixture can be stirred for a period of time to ensure the peptide becomes predominantly dissolved in the organic solvent. Therefore, upon mixing with the precipitating agent/coupling reagents, the peptide essentially is driven into the organic phase of the mixture.

Suitable solvents include halogenated organic solvents such as dichloropropane, dichloroethane (DCE), dichloromethane (DCM), chloroform, chlorofluorocarbons, chlorofluorohydrocarbons, and mixtures thereof. A preferred solvent is dichloromethane (DCM).

In some aspects, an organic solvent is used that can effectively keep the peptide in an organic phase in the presence of other aqueous solutions, such as water. In other aspects, an organic solvent is used that can be present as a cosolvent in a subsequent deprotection reaction. In yet other aspects, an organic solvent is used that has a low boiling point and that can be readily distilled without damage to the peptide. This allows the peptide to be concentrated in the organic solvent, by performing, for example, a distillation step.

The mixture can then be allowed to settle for a period of time to promote the separation of the organic phase from the phase that contains the precipitating agent. The phase that contains the precipitating agent can include other reagents present in the coupling reaction, such as the coupling solvent and coupling reagents. This phase that contains the precipitating agent can be removed from the reaction vessel.

According to the invention, the peptide can be kept dissolved in at least a portion of the organic solvent until it is introduced into the deprotection reaction. The organic solvent having the peptide can be kept at a temperature of, for example, less than 40° C., or more preferably, less than 20° C. This is advantageous, as it is not necessary to subject the peptide to a drying step prior to deprotection. This can reduce the possibility of peptide degradation, especially sensitive peptide fragments.

The organic solvent containing the peptide product can be washed one or more times with the precipitating agent or a similar solution. A suitable wash solution is a polar liquid, such as water. In some embodiments, the step of washing is repeated more than once, and preferably multiple times, in order to remove a reagent(s) or solvent(s) used in a previous step, for example, reagents and solvent from the coupling reaction. In the washing step, use of water is a very cost effective way to remove unwanted agents, such as those in the coupling reaction.

The wash steps can be carried out by adding wash solution to the organic solvent and peptide, mixing the organic solvent and wash solution, settling the mixture to allow the phases to separate, and then removing the wash solution. Any amount of wash solution can be used to wash the organic solvent and peptide; however it is preferable to use an amount of wash solution that is less than the amount of organic solvent, and more preferable less than half the amount of organic solvent as it can reduce time the overall wash times. In preferred embodiments steps in the wash process can be carried out at temperatures of 20° C. or lower.

After the final wash is separated from the organic solvent and removed, a distillation step, or a similar process, can be performed to concentrate the peptide in the organic solvent, if desired. The step of concentrating includes removing a portion of the organic solvent from the organic solvent/peptide mixture. The step of concentrating can also remove water present in the organic solvent and can therefore improve the deprotection reaction. The distillation process can be used if it is desired to provide the peptide to the deprotection reaction in a more concentrated form. For example, in some scaled-up embodiments it is preferred to have the peptide in a more concentrated form. In one embodiment of the invention, the peptide is concentrated in the organic solvent to 4 L/kg or less than 4 L/kg, for example in the range of 4 L/kg to 3 L/kg.

The distillation process can be performed at normal atmospheric pressures or by subjecting the organic solvent/peptide mixture to a vacuum of, for example, 250 mmHg or less. The distillation process can also be performed at a temperature of 25° C. or less. However, the actual vacuum and temperature may be varied, depending on the type of organic solvent used.

After the peptide is dissolved in an organic solvent it can be contacted with a deprotection agent. In some embodiments, the step of contacting includes preparing a deprotection composition and then mixing the deprotection composition with the organic solvent having the peptide. The organic solvent having the peptide and the deprotection composition can be combined in any suitable manner, for example, the organic solvent can be fed into a reaction vessel having the deprotection composition.

The actual volumes of organic solvent combined with the deprotection composition can depend on the concentration of peptide in the organic solvent and the concentration of the deprotection agent in the deprotection composition. In one embodiment, an amount of peptide is placed in contact with an amount of deprotection agent at a ratio of 1:8 or less based on weight, for example in the range of 1:8 to 1:16 based on weight. After the organic solvent/peptide mixture is mixed with the deprotection composition, the organic solvent is necessarily present as a cosolvent in the deprotection reaction.

The removal of side chain protecting groups by global deprotection typically utilizes a deprotection composition that includes an acidolytic agent to cleave the side chain protecting groups. Commonly used acidolytic reagents for global deprotection include triflouroacetic acid (TFA), HCl, lewis acids such as BF₃Et₂O or Me₃SiBr, liquid hydrofluoric acid (HF), hydrogen bromide (HBr), trifluoromethane sulfuric acid, and combinations thereof. The deprotection solution also includes one or more suitable chemical scavengers, for example, dithiothreitol, anisole, p-cresol, ethanodithiol, or dimethyl sulfide. The deprotection solution can also include water. As used herein, amounts of reagents present in the deprotection composition are typically expressed in a ratio, wherein the amount of an individual component is expressed as a numerator in “parts”, such as “parts weight” or “parts volume” and the denominator is the total parts in the composition. For example, a deprotection composition containing TFA:H₂O:DTT in a ratio of 90:5:5 (weight/weight/weight) has TFA at 90/100 parts by weight, H₂O at 5/100 parts by weight, and DTT at 5/100 parts by weight.

In one embodiment of the invention, the deprotection is carried out in the presence of the organic cosolvent and relatively high amounts of the acidolytic agent. According to the invention, the cosolvent also lends to the use of higher concentrations of the acidolytic agent, which can enhance the rate at which the peptide is deprotected. In some embodiments, the deprotection reaction can be performed wherein the amount of the acidolytic agent, preferably TFA, in the deprotection composition is greater than 90/100 parts by weight; 93/100 parts by weight or greater; or in the range of 93/100-95/100 parts by weight.

In other embodiments, the amount of water in the deprotection composition can be less than 5/100 parts by weight. More preferably the water is in an amount of 3.5/100 parts by weight or less. Most preferably, the water is in an amount in the range of 0.8/100 parts by weight to 3.5/100 parts by weight.

In addition, the amount of scavenger, preferably DTT, in the deprotection composition can be greater than 5/100 parts by weight.

Higher amounts of the acidolytic component, for example, TFA, in the deprotection composition allows the deprotection reaction to be performed more rapidly. Global deprotection can be performed, for example, in a large-scale peptide synthesis, for a period of 6.5 hours or less; preferably global deprotection is performed in the range of 5 to 6.5 hours. Temperatures for the global deprotection can be maintained in the range of 20° C.-30° C.

For example, presence of the cosolvent allows quenching of the deprotection reaction to be controlled to a greater extent. This, in turn, improves peptide quality. Also, presence of the cosolvent allows for better filtering and washing of the peptide after the deprotection reaction has been quenched. This, in turn, improves peptide quality and reduces processing time.

After the deprotection reaction is complete, the peptide can be precipitated with a precipitating agent. This step is also known as a quenching step as the precipitating agent effectively blocks the peptide from being further affected by the deprotection agent. Surprisingly, the presence of the organic cosolvent allows quenching of the deprotection reaction to be controlled to a greater extent. When the organic cosolvent is present residual activity of the deprotection agent is greatly reduced which thereby improves peptide quality.

The precipitating agent for the peptide, for example, an ethereal liquid, is added in an amount (“a precipitating amount”) sufficient to precipitate the peptide. One example of a precipitating agent is methyl-tert-butyl ether (MTBE). Another example of a precipitating agent is ether (diethyl) diisopropyl ether. The precipitating agent can be added to the solution having the peptide in the range of 4.7:10 to 12:10 Both the precipitating agent and the peptide solution can be combined at a temperature in the range of −5° C. to 5° C. In particular, temperatures of near −5° C. are preferred. In addition, the precipitating agent can be added at a rate of about 2 kg/min. After the cold solutions are combined, the mixture is gradually warmed, for example, to a temperature of 12° C. or greater.

After the precipitating agent has been blended and the peptide precipitate forms, a filter decanting step can be performed. Filter decanting is a particularly useful method for separating a precipitated peptide from the liquid that it is in. In filter decanting, a mixture containing a peptide precipitate is provided to a vessel having a filter decanting apparatus. In the case where a precipitation step is performed, the precipitation can be performed in the vessel having the filter decanting apparatus or the precipitation can occur in a different vessel and mixture containing the precipitate can be transferred to yessel having the filter decanting apparatus.

In addition, according to the invention, the presence of the organic cosolvent can improve the step of separating the liquid from the precipitated peptide.

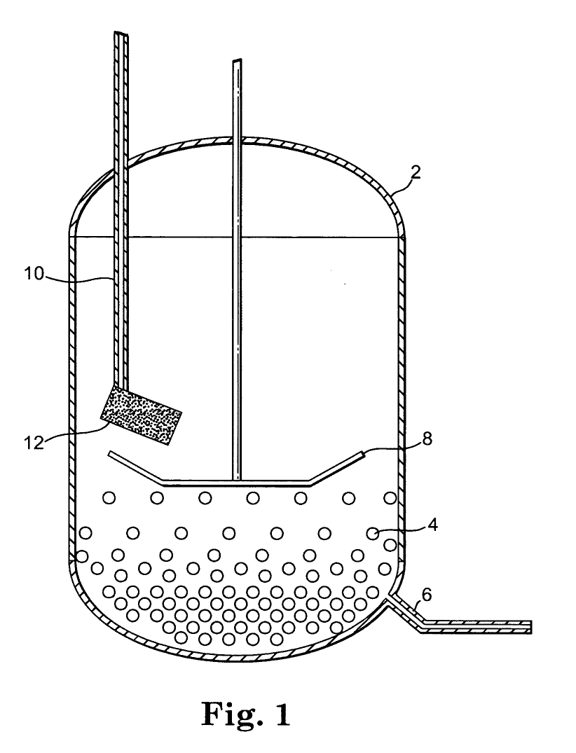

FIG. 1 illustrates a vessel 2 having a filter decanting apparatus. Generally, the vessel 2 contains a mixture that includes a precipitated peptide particle 4. In some cases, the mixture is formed by feeding a precipitating agent into the vessel 2, via a feed line 6. If a precipitating step is performed, an agitator 8 can be operated to blend the precipitating agent with the peptide. Parameters such as the temperature of the peptide in the vessel 2, the temperature of the precipitating agent, the feed rate of the precipitating agent, and the rate of the agitator 8 can be controlled either manually or automatically. Exemplary parameters are disclosed herein. An amount of precipitating agent can be added to the vessel 2 in order to precipitate the peptide to form a precipitated peptide particle 4. Additional precipitating agent can be added if desired.

Upon the addition of a precipitating amount of precipitating agent, the precipitated peptide 4 can settle by gravity in the vessel 2. The extent of settling can depend on a number of factors, including the peptide precipitate particle size and operational rate of the agitator 8. In some processes it may be desirable to allow the precipitate to settle to a certain extent before operating the filter decant apparatus.

When the precipitation step has proceeded to an acceptable point, the filter decanting process can be initiated. The filter decant method essentially removes most or all of the supernatant of the mixture from the vessel 2, leaving a peptide precipitate concentrate in the vessel. Filter decanting can be performed by extending tube 10 into the vessel 2, wherein the end of the tube 10 that is in contact with the supernatant has a decant filter 12. In order to remove the supernatant, a vacuum can be applied to tube 10 to suction off the liquid mixture through decant filter 12. If desired, the tube 10 and decant filter 12 can be raised or lowered during the process in order to place the decant filter 12 in a desired portion of the mixture. For example, during the beginning of the filter decant process the decant filter 12 can be placed in the upper levels of the mixture, where there can be a lower concentration of precipitated peptide and then gradually lowered as more mixture is removed from the vessel. As more mixture is removed, the precipitated peptide particle 4 remains in the lower portion of the vessel 2, on the sides of the vessel, or both. The decant filter 12 blocks most, if not all, of the precipitated peptide particle 4 from being removed with the liquid mixture. The agitator 8 can be operated during the filter decanting if so desired.

The dimensions of the decant filter 12 be sized in relation to the vessel. It is understood that by increasing the surface area on the surface of the decant filter 12 the flow rate of removal of supernatant from the vessel 2 can be increased. The decant filter 12 also includes a membrane having pores of a size that prevent most, if not all, of the precipitated peptide particle 4 from flowing through the decant filter 12.

In preferred embodiments the vessel 2, feed line 6, tube 10, and filter 12 are of a size to accommodate a scaled-up synthesis.

Following this filter decant step, the precipitated peptide particle 4 remaining in the vessel 2 can be washed once, or more than once, with a suitable liquid. Suitable liquids, for example the precipitating agent, typically keep the peptide in a precipitated form and are able to remove a portion or all of the remaining impurities. A desired number of washes can be performed at a desired temperature, amount of wash liquid, agitation rate, and time period. After each wash a step of filter decanting can be performed.

After the washes are performed, the cake of precipitated peptide particles can be dried or partially dried. In some embodiments, the precipitated peptide particles are partially dried and provided to a subsequent reaction as a wet cake. Drying can be performed for a relatively short period, for example, up to one hour.

In some embodiments, the peptide that has been subject to the precipitation and filter decanting steps as described herein can be subject to further chemical reactions or modifications. One exemplary modification is performing a decarboxylation reaction on the peptide. For example, in embodiments wherein the peptide is subject to a global deprotection reaction a peptide carbamate may be formed. After the peptide carbamate is taken through the precipitation and filter decanting methods as described herein, it can be subject to a decarboxylation step.

Decarboxylation can be performed using a decarboxylation reagent, such as acetic acid. Following the decarboxylation step, the peptide can again be precipitated and purified, for example, using the precipitation and decant filtration methods as described herein.

Additional procedures involved in the solid phase, solution phase, and/or hybrid synthesis of peptides are discussed in the following U.S. provisional applications: (1) U.S. provisional application No. 60/533,655, filed Dec. 31, 2003, titled “Methods For Recovering Cleaved Peptide From A Support After Solid Phase Synthesis” bearing attorney docket no. RCC0008/P 1, in the names of inventors including Robert Orr Cain; (2) U.S. provisional application No. 60/533,653, filed Dec. 31, 2003, titled “Process and Systems for Recovery of Peptides” bearing attorney docket no. RCC009/P1, in the names of inventors including Hiralal Khatri; (3) U.S. provisional application No. 60/533,691, filed Dec. 31, 2003, titled “Peptide Synthesis Using Filter Decanting” bearing attorney docket no. RCC0010/P1, in the names of inventors including Mark A. Schwindt; and (4) U.S. provisional application No. 60/533,654, filed Dec. 31, 2003, titled “Process and Systems for Peptide Synthesis” bearing attorney docket no. RCC0011/P1, in the names of inventors including Mark A. Schwindt.

The following non-limiting examples are provided to illustrate aspects of the present invention.

### EXAMPLE 1

### Preparation of Enfuvirtide

This example describes the formation and global deprotection of protected enfuvirtide using an organic cosolvent. This example also describes additional processing steps, including decarboxylation of the enfuvirtide carbamate. Four batches (A-D) of the peptide were prepared.

As starting material, a side-chain protected, N-terminal acetylated enfuvirtide peptide (Ac-AA(1-36)NH₂), having the formula: Ac-Tyr(tBu)-Thr(tBu)-Ser(tBu)-Leu-Ile-His(trt)-Ser(tBu)-Leu-Ile-Glu(OtBu)-Glu(OtBu)-Ser(tBu)-Gln(trt)-Asn(trt)-Gln(trt)- Gln-Glu(OtBu)-Lys(Boc)-Asn(trt)-Glu(OtBu)-Gln(trt)-Glu(OtBu)-Leu-Leu-Glu(OtBu)-Leu-Asp(tBu)-Lys(Boc)-Trp(Boc)-Ala-Ser(tBu)-Leu-Trp(Boc)-Asn(trt)-Trp(Boc)-Phe- NH2 (SEQ ID NO:1 with side chain protecting groups) was prepared using a combination of solid-phase and solution-phase peptide synthesis steps. Ac-AA(1-36)NH₂ can be prepared according to the methods described in U.S. Pat. No. 6,015,881.

Briefly, enfuvirtide peptide intermediate fragments H-AA(17-36)—NH2 (protected) was coupled to AC-AA(1-16)OH (protected) in a solution phase reaction to form Ac-AA(1-36)NH2 (protected). These solid phase synthesis and solution phase coupling steps to form the H-AA(17-36)—NH2 (protected) and Ac-AA(1-16)OH (protected) intermediate fragments can be performed according to the methods described in U.S. Pat. No. 6,015,881.

For each batch (batches A-D) the following procedure was carried out. The solid raw materials (Ac-AA(1-16)OH (protected); H-AA(17-36)—NH2 (protected); and 6-chloro-1-hydroxybenzotriazole (6-Cl-HOBT)) were charged to a vessel (see Table 2). The solids were dissolved in dimethylformamide (DMF) and the resulting solution was pre-cooled to 0° C. Diisopropylethylamine (DIEA) was added, followed by O-benzotriazol-1-yl-N,N,N′,N′-tetramethyluronium hexafluorophosphate (HBTU) as a solid. The reaction mixture was stirred at 0° C. then warmed to 20° C. over about 2 h (see Table 3; for the HPLC chromatogram, % AN (area norm) refers to the area percent under a peak at a particular wavelength). The reaction mixture was sampled and tested for the coupling reaction completion to the Ac-AA(1-36)NH₂ (protected) product. When complete, water was charged to the coupling mixture to precipitate Ac-AA(1-36)NH₂ (protected) as a solid (see Table 4). The solid product was dissolved and extracted into methylene chloride (see Table 5). The DMF/water layer was separated and removed. The methylene chloride solution of Ac-AA(1-36)NH₂ (protected) was washed with water several times to remove residual DMF. The Ac-AA(1-36)NH₂ (protected) solution was concentrated by vacuum distillation to remove most of the methylene chloride (see Table 6). The target volume of remaining methylene chloride was 3-4 L/kg of peptide. The jacket temperature was kept below 45° C. and the vessel temperature was kept below 40° C. during the distillation. The concentrated methylene chloride solution of Ac-AA(1-36)NH2 (protected) was added to a mixture of trifluoroacetic acid (TFA), dithiothreitol and water (in a 94.0:5.2:0.8 w/w/w ratio) (see Table 7). The de-protection reaction mixture was stirred at 25° C. (±3) for 4-6 h, and cooled to 0° C. (±5). MTBE was charged to quench the reaction and to precipitate Ac-AA(1-36)NH2 carbamate (see Table 8). The solids were filtered, washed with MTBE, and partially dried up to 60% LOD. In all batches, a Hasteloy 10-20 μm filter was used to isolate Ac-AA(1-36)NH₂ carbamate by a filter decanting process (apparatus and method in accordance with FIG. 1; see also Table 9). The wet cake was washed with MTBE three times.

Isopropanol, DIEA, and acetic acid were charged to the semi dried solid intermediate in the decant vessel to obtain a pH typically between 4 and 5 (see Table 11). The slurry mixture was warmed to 35° C. (±5), sampled, and tested for de-carboxylation reaction completion. The slurry mixture was cooled to 10° C. (+5), the solid Ac-AA(1-36)NH2 product filtered, and washed with isopropanol. In all batches, a Heinkel filter with polypropylene filter cloth, 5-7 μm size, was used to isolate the Ac-AA(1-36)NH₂ product (see Table 12). Isolation involved filtering to remove the liquors, followed by washing with isopropanol. The cake was then blown down to remove as much isopropanol as possible from the cake. The Ac-AA(1-36)NH₂ product was dried under 10 vacuum and packaged (see Table 13). DV-111 rotary dryer was used as the dryer for preparation of batches A-D. The wet cake from Heinkel isolation was dried under vacuum at less than 40° C. until the LOD was less than 2.0%. Data for purity and yield is provided in Table 14.

TABLE 2
Raw Material Loading Data

| | Batch | | | |
|---|---|---|---|---|
| | A | B | C | D |
| Ac-AA(1-16)OH charge, (kg) | 9.0 | 8.6 | 8.6 | 8.4 |
| Ac-AA(1-16)OH, (mol) | 2.73 | 2.61 | 2.61 | 2.55 |
| H-AA(17-36)NH₂ charge, (kg) | 11.4 | 11.4 | 11.4 | 11.4 |
| H-AA(17-36)NH₂, (mol) | 2.75 | 2.75 | 2.75 | 2.75 |
| 6-Chloro-HOBt charge, (kg) | 0.8 | 0.7 | 0.7 | 0.7 |
| DIEA charge, (kg) | 0.65 | 0.65 | 0.65 | 0.65 |
| HBTU charge, (kg) | 1.2 | 1.2 | 1.2 | 1.2 |
| DMF charge, (kg) | 114.0 | 114.0 | 113.9 | 113.1 |

TABLE 3
Coupling Reaction Data

Batch

| | A | B | C | D |
|---|---|---|---|---|
| Stir time, (h:min) | 10:15 | 2:0 | 1:11 | 1:15 |
| Warm up time to 25° C., | N/A | 0:40 | 0:23 | 0:33 |
| (h:min) | | | | |
| Age time before quench, | N/A | 2:0 | 2:0 | 2:0 |
| (h:min.) | | | | |

Coupling Reaction Completion to Ac-AA(1-36)NH₂ HPLC
Results, (% AN)

| | | | | |
|---|---|---|---|---|
| Ac-AA(1-36)NH₂ | 68.9 | 57.6 | 69.8 | 68.8 |
| Ac-AA(1-16)OH | 2.7 | 0.76 | 0.5 | 1.6 |
| H-AA(17-36)NH₂ | 0.1 | 0.11 | 0.9 | 0.6 |

TABLE 4
Water Quench Data

| | Batch | | | |
|---|---|---|---|---|
| | A | B | C | D |
| Water charge (L) | 120 | 120 | 120 | 120 |
| Water addition time (min) | 31 | 31 | 50 | 30 |
| Exothermic range (° C.) | 5-17 | 18-25 | 16-22 | 20-21 |

TABLE 5
Extraction Data

| | Batch | | | |
|---|---|---|---|---|
| | A | B | C | D |
| Methylene chloride charge (kg) | 425.0 | 426.0 | 425.0 | 429.0 |
| Stir period (min) | 15 | 15 | 15 | 30 |
| Stir temperature (° C.) | 14 | 14-15 | 15-16 | 12-13 |
| Settle period (min) | 47 | 130 | 125 | 120 |
| Settle temperature (° C.) | 14-15 | 14-15 | 16-18 | 13-14 |
| Water charge #1 (L) | 120.0 | 120.0 | 158.0 | 158.0 |
| Stir period (min) | 15 | 15 | 15 | 15 |
| Stir temperature (° C.) | 16-17 | 19 | 19 | 16-17 |
| Settle period (min) | 45 | 83 | 45 | 55 |
| Settle temperature (° C.) | 17 | 18-19 | 19 | 17-18 |
| Water charge #2 (L) | 120.0 | 120.0 | 158.0 | 158.0 |
| Stir period (min) | 16 | 15 | 17 | 15 |
| Stir temperature (° C.) | 16 | 16 | 17 | 16 |
| Settle period (min) | 138 | 45 | 105 | 360* |
| Settle temperature (° C.) | 16 | 16 | 17-18 | 16-18 |
| Water charge #3 (L) | 120.0 | 120.0 | 158.0 | 158.0 |
| Stir period (min) | 15 | 16 | 16 | 15 |
| Stir temperature (° C.) | 16 | 18 | 17-18 | 17-18 |
| Settle period (min) | 61 | 49 | 49 | 45 |
| Settle temperature (° C.) | 16 | 18-19 | 17-18 | 17-18 |
| Water charge #4 (L) | 120.0 | 120.0 | 158.0 | 158.0 |
| Stir period (min) | 15 | 15 | 15 | 81 |
| Stir temperature (° C.) | 15 | 17 | 16 | 18-23 |
| Settle period (min) | 45 | 47 | 45 | 320 |
| Settle temperature (° C.) | 15 | 17-18 | 16-17 | 23-24 |
| Water charge #5 (L) | 120.0 | 120.0 | 158.0 | 158.0 |
| Stir period (min) | 15 | 15 | 15 | 15 |
| Stir temperature (° C.) | 15 | 17-19 | 16-17 | 20-23 |
| Settle period (min) | 46 | 70 | 120 | 324 |
| Settle temperature (° C.) | 15-16 | 17-19 | 16-18 | 20-24 |
| Gas Chrom. % w/w DMF | 2.5 | 1.8 | 1.1 | 1.5 |
| DCM volume, L | 300 | 340 | 320 | 400 |

Batch D had an emulsion problem during the 4^{th} water wash. This emulsion was due to a slight increase in agitation speed. The problem was solved by extending settle time, increasing the batch temperature and by taking emulsified layer with organic layer and adding more methylene chloride to separate the layers. Excess methylene chloride was removed by distillation.

TABLE 6
Distillation Data

| | Batch | | | |
|---|---|---|---|---|
| | A | B | C | D |
| Distillation time, h:min | 20:15* | 15:15* | 03:38 | 05:18 |
| Distillation temperature, (° C.) | 6-23 | 6-19 | 4-18 | 11-24 |
| Vacuum, (mmHg) | 150 | 150 | 170 | 230 |
| Batch Volume, (L) | 70 | 70 | 70 | 70 |

Distillate Sample Results

| | | | | |
|---|---|---|---|---|
| Ac-AA(1-36)NH₂, (g/L) | 4.1 | 1.8/0.47 | 5.3 | 0.1 |
| Distillate volume, (L) | 230 | 240/260 | 250 | 260 |

Distillation Completion Sample Results

| | | | | |
|---|---|---|---|---|
| Water, (%) | 1.13 | 0.59 | 0.08 | 0.05 |
| DMF, (wt./wt. %) | 10.2 | 5.6 | 3.7 | 5.6 |
| DCM volume, L | 70 | 70 | 70 | 70 |
| DCM charge, kg | 350 | 350 | N/A | N/A |
| GC % w/w Results for DMF | 4.4 | 6.2 | | |
| KF, % water | 0.37 | 0.022 | | |
| DCM volume, L | 70 | 70 | | |

*DCM addition and extraction repeated due to higher DMF levels after first distillation.

TABLE 7
De-protection Reaction Data

| | Batch | | | |
|---|---|---|---|---|
| | A | B | C | D |
| Trifluoroacetic acid charge (kg) | 189.1 | 244.8 | 244.9 | 247.3 |
| Dithiothreitol charge (kg) | 10.2 | 13.6 | 13.4 | 13.6 |
| Potable water charge (kg) | 4.0 | 2.0 | 2.0 | 2.0 |
| DCM rinse (kg) | 37.0 | 37.0 | 37.0 | 32.0 |
| Reaction time (h) | 10 | 6 | 5 | 5 |
| Stir temperature (° C.) | 16-23 | 26 | 25 | 22 |

Rxn completion, Ac-AA(1-36)NH₂, carbamate

| | | | | |
|---|---|---|---|---|
| HPLC % AN Results for | 61.5 | 63.1 | 72.3 | 70.3 |
| Ac-AA(1-36)NH₂ | | | | |
| % AN for 9.5 (RRT 1.23) peak | 2.7 | 1.5 | 0.6 | 1.7 |

TABLE 8
MTBE Quench Data

| | Batch | | | |
|---|---|---|---|---|
| | A | B | C | D |
| MTBE Charge (kg) | 318.8 | 318.4 | 319.0 | 318.0 |
| MTBE Temperature (° C.) | 0 (±3) | 0 (±3) | 0 (±3) | 0 (±3) |
| Batch temperature prior to quench | −4 | 1 | −5 | −5 |
| (° C.) | | | | |
| Transfer rate (kg/min) | 0.3-1.7 | 2.0-2.5 | 2.0 | 1.8-2.0 |
| Jacket heat up ramp to | 7 | 15 | 15 | 15 |
| 15° C., ° C./hr | | | | |
| Total Feed time (h:min) | 4:37 | 2:28 | 3:0 | 3:0 |
| Age time (min) | 30 | 30 | 30 | 35 |

MTBE was added through a mass flow meter and the bath was ramped during the feed/precipitation.

TABLE 9
Decant Filtration Data

Batch

| | A | B | C | D |
|---|---|---|---|---|
| Initial Filtration time, | 7:00 | 5:16 | 3:45 | 3:00 |
| (h:min) | | | | |
| 1^{st} MTBE charge for | 60.5 | 59.7 | 60.6 | 60.5 |
| washing (kg) | | | | |
| 2nd MTBE wash charge | 60.5 | 57.0 | 60.8 | 60.5 |
| (kg) | | | | |
| 3rd MTBE wash charge | 60.4 | 63.6 | 60.5 | 57.3 |
| (kg) | | | | |
| Total decant wash time | 6:10 | 9:30 | 5:24 | 8:50 |
| h:min | | | | |

TABLE 10
Drying Data

Batch

| | A | B | C | D |
|---|---|---|---|---|
| Jacket temperature (° C.) | −5 | −5 | −3 | −3 |
| Drying time (h:min) | 0:45 | 0:50 | 0:33 | 0:30 |
| Final LOD (%) | 47.63 | 60.23 | 57.24 | 56.98 |

TABLE 11
De-carboxylation Reaction Data

| | Batch | | | |
|---|---|---|---|---|
| | A | B | C | D |
| Isopropanol charge (kg) | 145.2 | 218.0 | 220.3 | 221.8 |
| DIEA charge (kg) | 5.3 | 8.0 | 7.9 | 8.0 |
| Acetic acid charge (kg) | 11.0 | 11.0 | 11.1 | 11.0 |
| Batch pH | 4.8 | 4.5 | 4.99 | 5.08 |

Rxn Time, h:min @ Temperature ° C.

| | | | | |
|---|---|---|---|---|
| Warm up | 6:02 @ 0-35 | 1:10 @ 14-30 | 1:0 @ 14-30 | 0:30 @ 19-33 |
| Age | * | 1:10 @ 32 | 1:05 @ 30-31 | 0:05 @ 33-34 |

*Not recorded

TABLE 12
Heinkel Filtration Data

| | Batch | | | |
|---|---|---|---|---|
| | A | B | C | D |
| Isopropanol charge (kg) | 200.1 | 203.6 | 77.9 | |
| Total # of spins | 52 | 53 | 64 | 46 |
| Wet cake (kg) | 28.6 | 27.5 | 29.8 | 26.9 |
| Filtration time (h:min) | 10:00 | 10:28 | 16:00 | 07:50 |
| Product loss in mother | 0.04 | 0.03 | 0.02- | 0.06 |
| liquors (g/L) | | | | |
| Volume L | 635 | 400 | 600 | 430 |

TABLE 13
Drying Data

| | Batch | | | |
|---|---|---|---|---|
| | A | B | C | D |
| Average jacket | 38 | 38 | 38 | 38 |
| temperature (° C.) | | | | |
| Vacuum (inch Hg) | 9-22 | 6-20 | 8-22 | 8-30 |
| LOD (%) | 0.32 | 0.68 | 0.56 | 0.75 |
| Drying time (d:h:min) | 04:06:25 | 02:16:11 | 03:07:05 | 02:15:03 |

TABLE 14
Yields and Purity Data

| | Batch | | | |
|---|---|---|---|---|
| | A | B | C | D |
| Yield Ac-AA(1-36)NH₂ (%) | 92.2 | 94.7 | 105.2 | 99.5 |
| Quantity of Ac-AA(1-36) | 11.4 | 11.7 | 13.0 | 12.3 |
| NH₂ (kg) | | | | |
| Purity (% AN) | 58.1 | 61.1 | 70.6 | 68.5 |
| Purity (% w/w) | 44.8 | 43.8 | 50.8 | 51.3 |
| Contained Yield, | 5.11 | 5.13 | 6.60 | 6.31 |
| (y × wt assay), kg | | | | |

1. A method for deprotecting a peptide comprising steps of:
(a) providing a peptide composition comprising i) a non-resin bound peptide material comprising at least one protecting group and ii) an organic solvent; and
(b) contacting the peptide composition with a deprotection agent.

2. The method of claim 1 wherein the organic solvent comprises a halogenated liquid.

3. The method of claim 1 wherein the halogenated liquid comprises DCM.

4. The method of claim 1 wherein the non-resin bound peptide material comprises a side chain protecting group.

5. The method of claim 1 further comprising a step of removing a portion of the organic solvent by distillation to form the peptide composition.

6. The method of claim 5 wherein the distillation is performed at a temperature of less than 45° C.

7. The method of claim 4 wherein the distillation is performed to provide the non-resin bound peptide material at a concentration of 4 L/kg or less in the organic solvent.

8. The method of claim 1 comprising a step of washing the peptide composition with a precipitating agent, the step of washing being performed before step (b).

9. The method of claim 1 wherein the precipitating agent is water.

10. The method of claim 1 comprising a step of coupling in solution i) an amino acid to an amino acid; ii) an amino acid to a peptide intermediate; or iii) a peptide intermediate to a peptide intermediate, to form the non-resin bound peptide material.

11. The method of claim 10 wherein a step of drying the non-resin bound peptide material is not performed between the step of coupling and the step of contacting the peptide composition with a deprotection agent

12. The method of claim 1 wherein step (b) the deprotection agent comprises an acidolytic compound present in a deprotection composition, the deprotection composition further comprising water and a scavenger.

13. The method of claim 12 wherein the acidolytic compound comprises TFA and wherein the TFA is present in the deprotection composition at greater than 90/100 parts by weight.

14. The method of claim 13 wherein the TFA is present in the deprotection composition at 93/100 parts by weight or greater.

15. The method of claim 14 wherein the TFA is present in the deprotection composition at 95/100 parts by weight or greater.

16. The method of claim 13 wherein the TFA is present in the deprotection composition in the range of 93/100 parts by weight to 95/100 parts by weight.

17. The method of claim 12 wherein the water is present in the deprotection composition at less than 5/100 parts by weight.

18. The method of claim 17 wherein the water is present in the deprotection composition at 3.5/100 parts by weight or less.

19. The method of claim 18 wherein the water is present in the deprotection composition in the range of 3.5/100 parts by weight to 0.8/100 parts by weight.

20. The method of claim 12 wherein the scavenger is DTT and wherein DTT is present in the deprotection composition at greater than 5/100 parts by weight.

21. The method of claim 1 wherein the peptide composition is not subject to a step of drying immediately prior to step (b).

22. The method of claim 1 wherein the non-resin bound peptide material comprises SEQ ID NO 1.

23. A method for preparing and deprotecting a peptide comprising steps of:
(a) forming a peptide material comprising a protecting group in a solution phase reaction;
(b) dissolving the peptide material in an organic solvent to form a peptide composition; and
(c) contacting the peptide composition with a deprotection agent.

24. A method for deprotecting a peptide comprising steps of:
(a) concentrating a non-resin bound peptide material comprising a protecting group in an organic solvent to provide a peptide composition; and
(b) contacting the peptide composition with a deprotection agent.
